(19) United States
(12) Patent Application Publication
(10) Pub. No.: US 2009/0281629 A1
(43) Pub. Date: Nov. 12, 2009

(54) Intervertebral disc prosthesis

(76) Inventors: Christian Roebling, Freiburg (DE); Rolf Ackermann, Rietheim-Weilheim (DE)

Correspondence Address: THE NATH LAW GROUP, 112 South West Street, Alexandria, VA, 22314, US

(21) Appl. No.: 12/453,248

(22) Filed: May 4, 2009

(30) Foreign Application Priority Data
May 5, 2008 (EP) 08 008423.9

(51) Int. Cl. A61F 2/44

(52) U.S. Cl. 623/17.16; 623/17.14

(57) ABSTRACT

The invention concerns an intervertebral disc prosthesis (10) with a first prosthesis plate (20) and a second prosthesis plate (30), wherein the first prosthesis plate (20) has on its side (20b) facing the second prosthesis plate (30) a concave recess (22) in which engages a convex projection (32) arranged on the side (30b) of the second prosthesis plate (30) facing the first prosthesis plate (10), wherein the convex projection (32) and/or the concave recess has at least one elastic element (35).

The invention concerns an intervertebral disc prosthesis according to the preamble of Claim 1.

Two-component or multicomponent intervertebral disc prostheses are known, wherein a hinge is formed between at least two parts to better simulate the possible movements of the intervertebral disc by the intervertebral disc prosthesis. Particularly, intervertebral disc prostheses with a first prosthesis plate and a second prosthesis plate are known, wherein the first prosthesis plate has, on its side facing the second prosthesis plate, a concave recess in which engages a convex projection arranged on the side of the second prosthesis plate facing the first prosthesis plate, so that, in this manner, a type of ball-and-socket joint is formed. When inserting such intervertebral disc prostheses, the intermediate space between two adjacent discs usually must first be spread apart.

The known intervertebral disc prostheses are usually made from two prosthesis plates made from a metal or a prosthesis plate made from metal and a prosthesis plate made from plastic, wherein the distance between the two prosthesis plates is defined.

The problem of the invention consists in disclosing an intervertebral disc prosthesis that better simulates the intervertebral disc to be replaced.

The problem is solved according to the invention by an intervertebral disc prosthesis with the features of Claim 1.

The intervertebral disc prosthesis according to the invention with a first prosthesis plate and a second prosthesis plate, wherein the first prosthesis plate has, on its side facing the second prosthesis plate, a concave recess in which engages a convex projection arranged on the side of the second prosthesis facing the first prosthesis plate, is designed such that the convex projection and/or the concave recess has at least one elastic element. The elastic element allows a relative movement of the two prosthesis plates against one another in the vertical direction. The elastic element has the advantage that it acts as a damping element, so that the function of the defective intervertebral disc is better simulated.

The convex projection and/or the concave recess has an especially preferred hollow configuration, by means of which a certain spring effect or elasticity of the convex projection is already generated that allows a relative movement between the two prosthesis plates and creates a damping effect.

The convex projection and/or the concave recess has at least an especially preferred slot by means of which, in particular, at least two elastic elements are formed, so that, for an elevated pressure, the convex projection can be pressed deeper into the concave recess of the first prosthesis plate. Here, a slot can be understood to be made from openings that have approximately the same longitudinal extent in two directions that are perpendicular to each other. In addition, the shape of the slots could also be arbitrarily varied. The slot should merely promote a spring effect of the projection or the recess. The slots could have profiles that are straight, curved, or curved multiple times, such as, for example, with a wave or also spiral shape.

The slots are preferably cut at an angle to the surface of the convex projection or the concave recess, so that, under a large load, the side edges of adjacent elastic elements come to lie not only against each other, but also one on top of the other in the radial direction, which increases the stability of the projection or the recess.

According to an especially preferred embodiment of the invention, the convex projection and/or the concave recess has, starting from its pole, i.e., from the highest point of the curved surface, several slots running in the direction of the edge of the convex projection. In this way, several elastic elements are formed that allow especially good damping.

To maintain the best possible movement of two adjacent discs against one another, the projection can preferably rotate and pivot in the recess.

According to an especially preferred execution, the projection has a spherical segment design to allow, in particular, rotation and tilting in any arbitrary direction. The recess preferably has a spherical shell segment design to be able to correspond especially well to the projection. Alternatively, in a preferred embodiment of the invention, the recess has an ellipsoidal shell segment design to allow a linear movement, particularly in the sagittal direction, in addition to the rotation and tilting of the two prosthesis plates against one another. The longitudinal axis of the ellipsoidal shell segment recess preferably lies in the sagittal direction.

To optimally map the natural center of rotation of the intervertebral disc, according to a preferred embodiment of the invention, the projection is arranged offset in the sagittal direction toward the back from the center of the prosthesis plate.

According to a preferred embodiment of the invention, between the first prosthesis plate and the second prosthesis plate, there is a guide element that has a base body and a head arranged on one end of the base body, wherein the base body engages in a guide recess in the second prosthesis plate and the head engages in a guide recess in the first prosthesis plate. The guide bolt forms subluxation protection, especially for non-biological movements or whiplash injury. For this purpose, however, the guide element is preferably guided such that it rarely negatively affects the other possible movements of the two prosthesis plates against one another. Another advantage of such a guide element lies in that it is used as a depth stop for the elastic element to limit the elastic movement in the axial direction.

The guide recess in the second prosthesis plate is preferably arranged in the projection, particularly between the elastic elements, to lie essentially in the vicinity of the rotational axis of the intervertebral disc prosthesis.

The guide recess of the second prosthesis plate preferably fixes the guide element in the horizontal and vertical direction relative to the second prosthesis plate. As a result, the guide element is fixed on a prosthesis plate.

The guide recess of the first prosthesis plate is preferably arranged in the concave recess to also allow in this prosthesis plate the arrangement of the guide element in the vicinity of the rotational axis of the intervertebral disc prosthesis.

The guide recess of the first prosthesis plate preferably allows movement of the guide element in the horizontal and vertical direction. Particularly in combination with the fixing of the guide element in the horizontal and vertical direction relative to the second prosthesis plate by the guide recess of the second prosthesis plate, the guide element barely limits the relative movement of the two prosthesis plates against one another.

Optionally, to allow a linear movement in the sagittal direction in combination with an ellipsoidal shell segment recess, the guide recess of the first prosthesis plate preferably has an oval or elliptical shape, wherein the longitudinal axis of the guide recess runs in the sagittal direction.

According to a preferred recess form [sic; embodiment] of the invention, the first prosthesis plate and/or the second prosthesis plate is made from PEEK (polyether ether ketone) that features good biocompatible properties. A special advantage of this material is that it is both CT (computer tomography) and also MRT (magnetic resonance tomography) compatible and does not generate artifacts in the created images like, in particular, various metals.

The side of the first and/or second prosthesis plate facing the disc preferably has an osteoconductive and/or an osteoinductive coating to allow the quickest possible and longest lasting biological integration of the prosthesis in the body.

According to an especially preferred embodiment of the invention, on the side facing the disc of at least one of the prosthesis plates, there is at least one anchoring element for anchoring the prosthesis plate in the adjacent disc, wherein the anchoring element can be countersunk in the prosthesis plate and moved at least partially out of the prosthesis plate. As a result, it is possible to insert the intervertebral disc prosthesis with initially countersunk anchoring elements into the intermediate space between two adjacent discs, wherein the intermediate space must be spread apart less than would be the case for anchoring elements rigidly arranged on the side of the prosthesis plate facing the discs. When the intervertebral disc prosthesis is correctly positioned, the anchoring elements are moved out from the prosthesis plate and, thus, contact the adjacent disc to fix the intervertebral disc prosthesis.

The anchoring element is preferably arranged in an anchoring recess of the prosthesis plate and can be moved at least partially out from the anchoring recess of the prosthesis plate by an activation element. Here, the activation element could be similarly arranged on the intervertebral disc prosthesis or could be formed as a separate tool that is then removed from the intervertebral disc space.

The activation element preferably has a forced surface on which runs a support edge of the anchoring element when the activation element moves, to generate, in this manner, the movement of the anchoring element, or at least a part of this anchoring element, out from the receptacle recess of the prosthesis plate. The forced surface preferably has a wedge- or cone-shaped design.

An especially simple configuration of anchoring elements is produced when the anchoring element has a U-shaped design, wherein a free end of one of the legs of the U-shaped anchoring element contacts the forced surface of the activation element that runs in this manner across the forced surface when the activation element moves, such that it is guided out from the anchoring recess of the prosthesis plate.

According to an especially preferred embodiment of the invention, the anchoring element has a catch element that, in the position moved out from the recess, interacts with a catch element of the activation element in this manner to prevent the anchoring elements from possibly sliding back into the anchoring recess after introducing the intervertebral disc prosthesis into the intervertebral disc space and extending the anchoring elements, producing the risk that the intervertebral disc prosthesis could become loose.

The catch element of the anchoring element is preferably designed as a catch projection arranged on a free end of one of the legs of the U-shaped anchoring element and the catch element of the activation element as a peripheral groove arranged in connection to the wedge- or cone-shaped forced surface, which allows an especially simple catch connection.

The invention will be explained in greater detail with reference to the following figures.

Shown are:

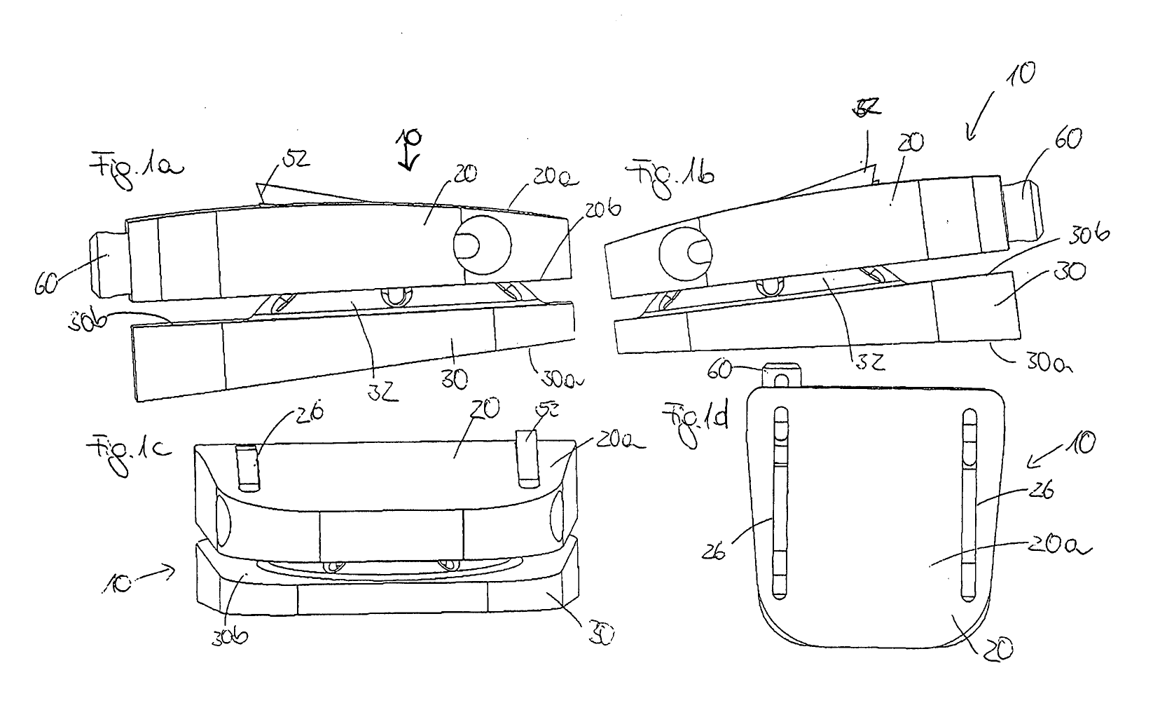

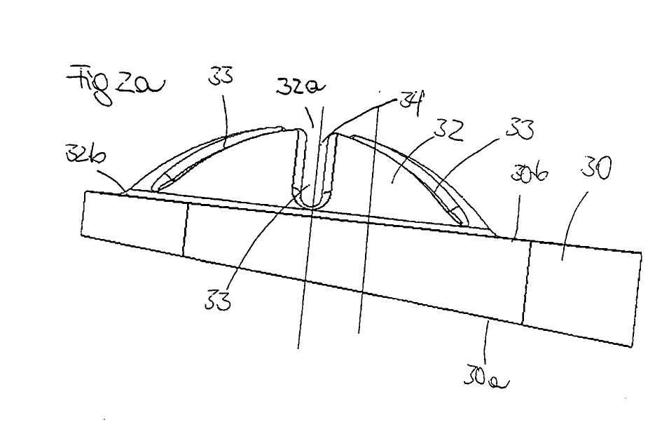

FIG. 1a, a side view of a first embodiment example of an intervertebral disc prosthesis with a first prosthesis plate and a second prosthesis plate,

FIG. 1b, another side view of the intervertebral disc prosthesis according to FIG. 1a,

FIG. 1c, a perspective view of the intervertebral disc prosthesis according to FIG. 1a,

FIG. 1d, a top view of the intervertebral disc prosthesis according to FIG. 1a,

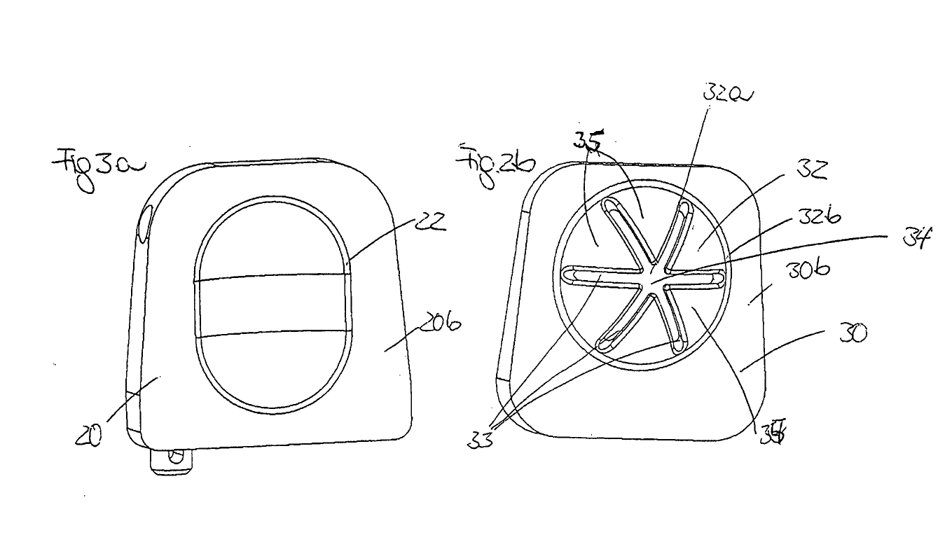

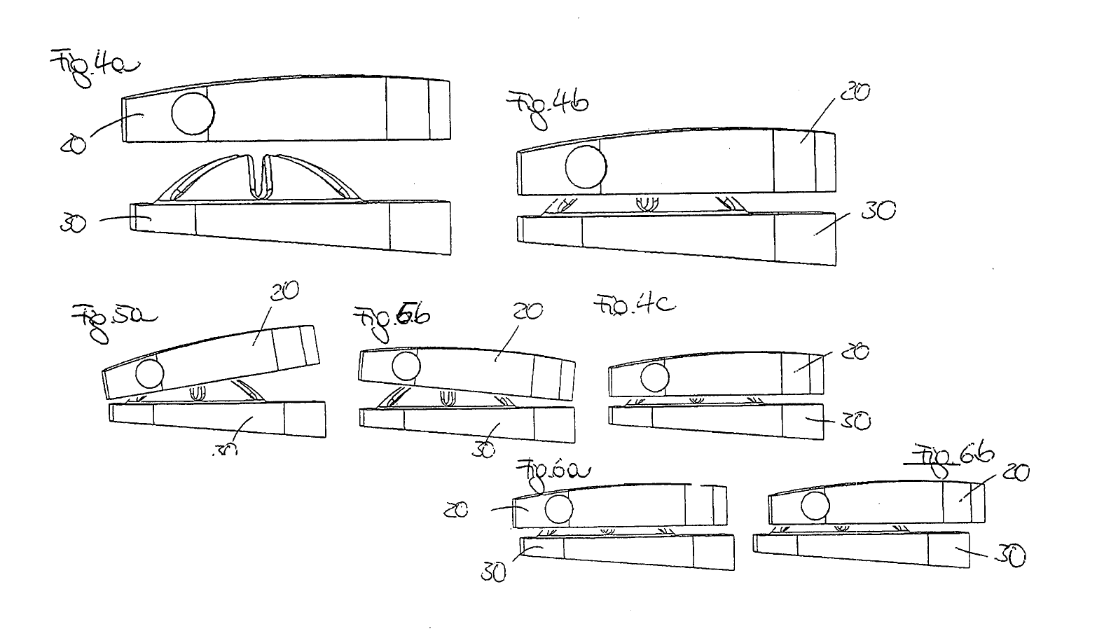

FIG. 2a, a side view of the second prosthesis plate of the intervertebral disc prosthesis according to FIG. 1a,

FIG. 2b, a perspective view of the prosthesis plate according to FIG. 2a,

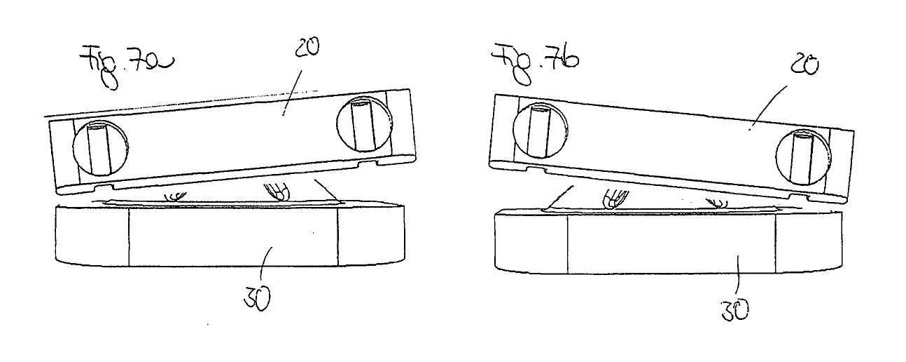

FIG. 3a, a perspective view of the first prosthesis plate of the intervertebral disc prosthesis according to FIG. 1a,

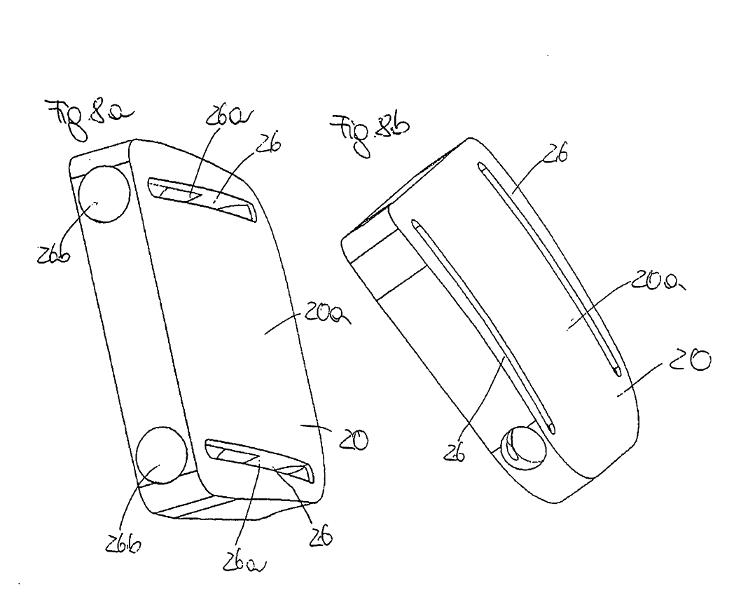

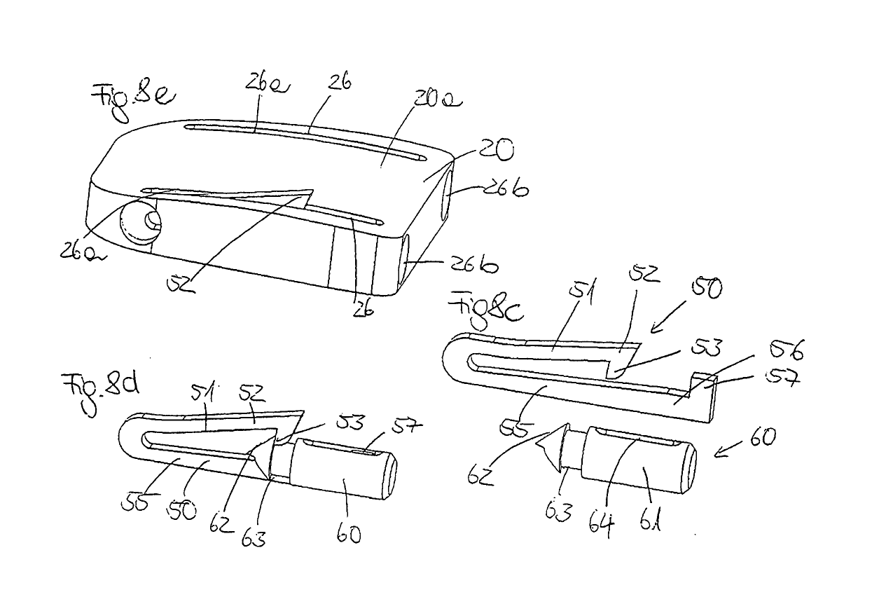

FIG. 4a, the two prosthesis plates of the intervertebral disc prosthesis according to FIG. 1a with detached first prosthesis plate,

FIG. 4b, the intervertebral disc prosthesis according to FIG. 4a with mounted first prosthesis plate,

FIG. 4c, the intervertebral disc prosthesis according to FIG. 4a in the flattened position,

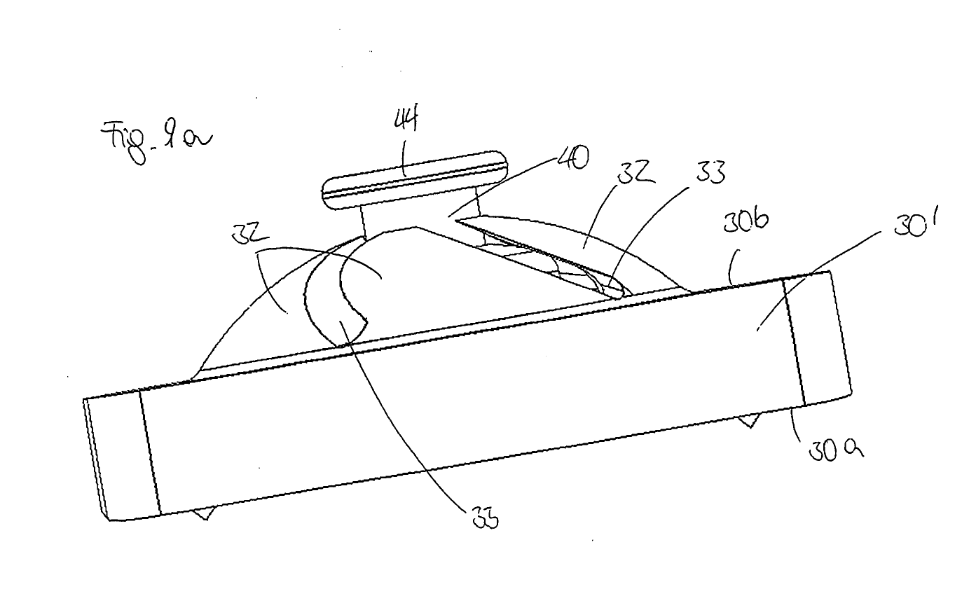

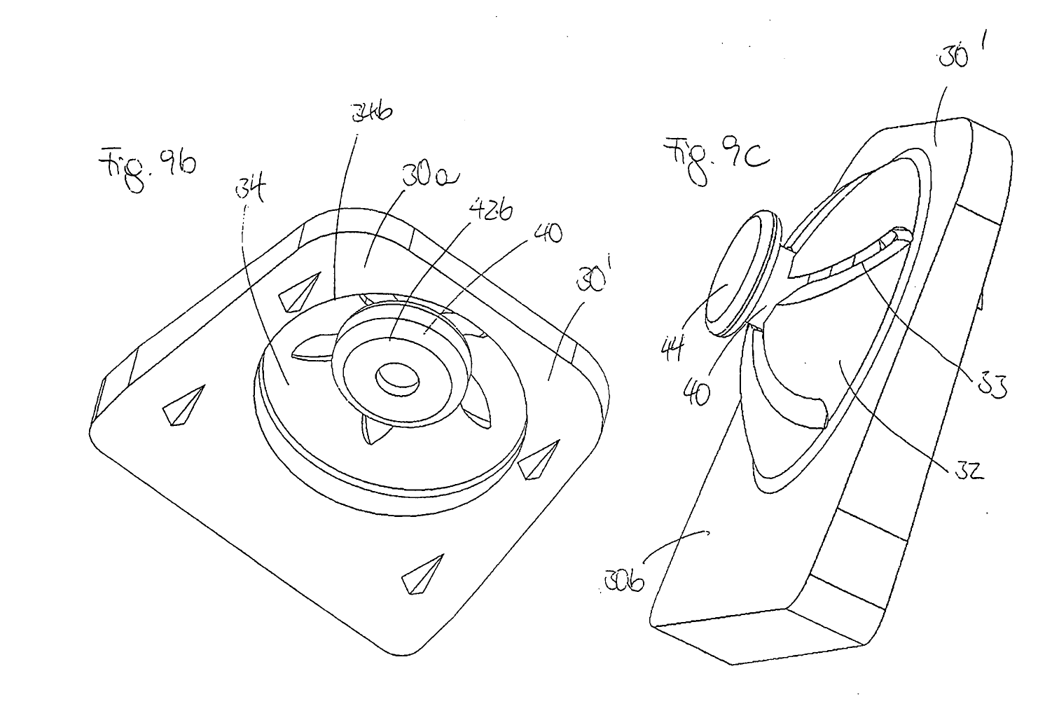

FIG. 5a, a tilted position of the intervertebral disc prosthesis according to FIG. 1a,

FIG. 5b, another tilted position of the intervertebral disc prosthesis according to FIG. 1a,

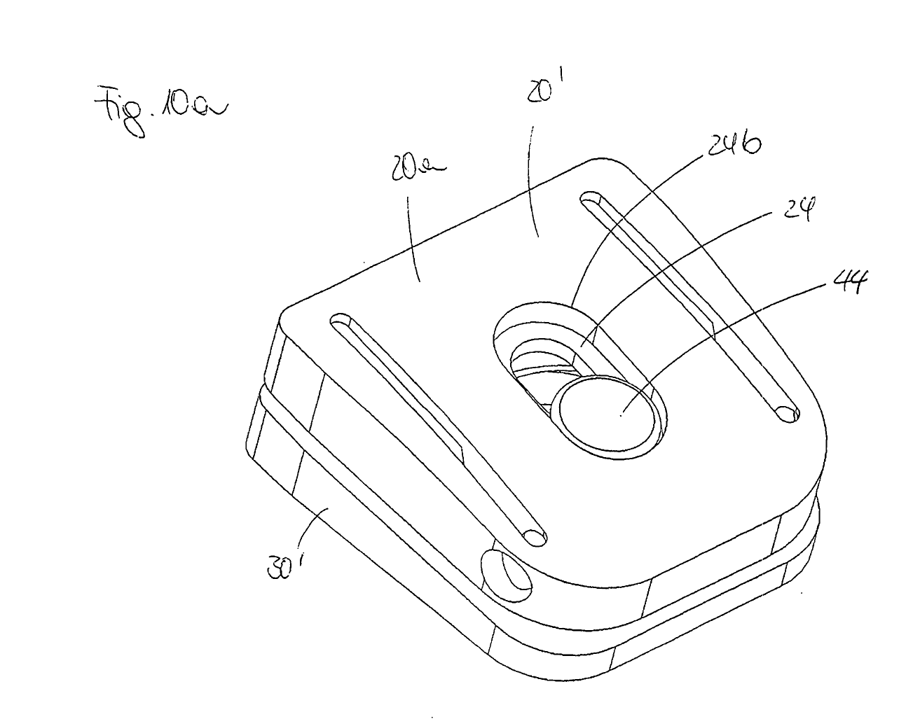

FIG. 6a, the intervertebral disc prosthesis according to FIG. 1a with first prosthesis plate shifted in a linear direction,

FIG. 6b, the intervertebral disc prosthesis according to FIG. 1a with the first prosthesis plate in another position shifted in a linear direction,

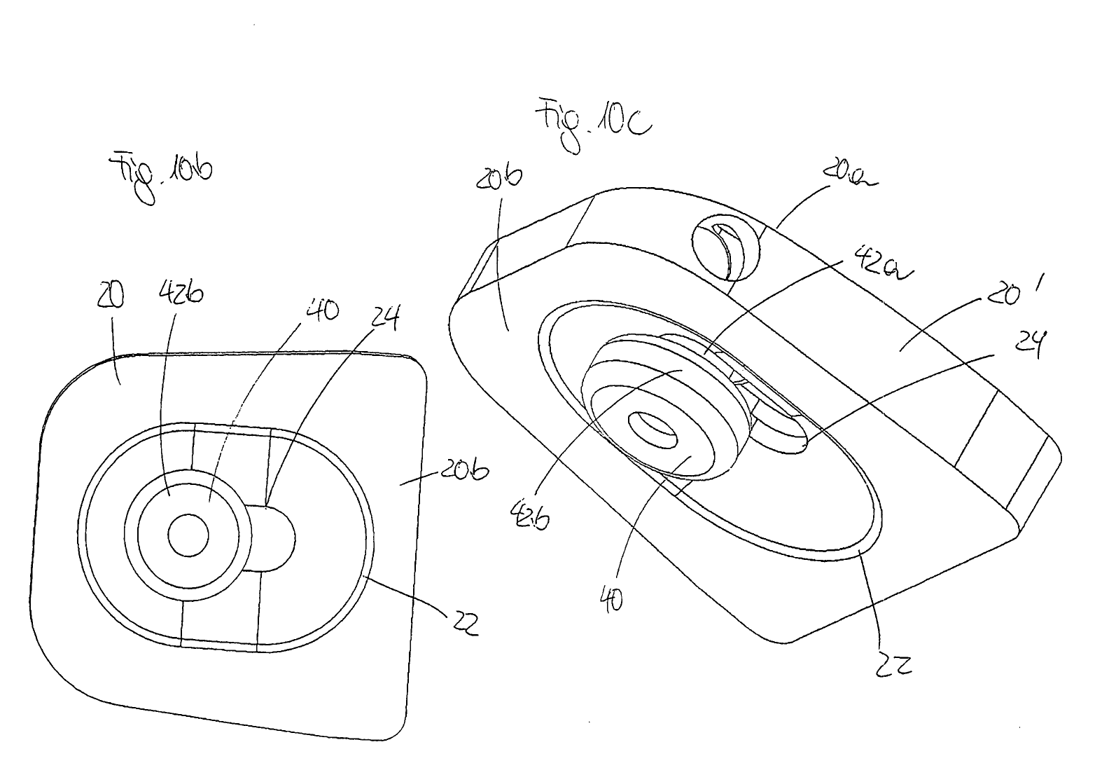

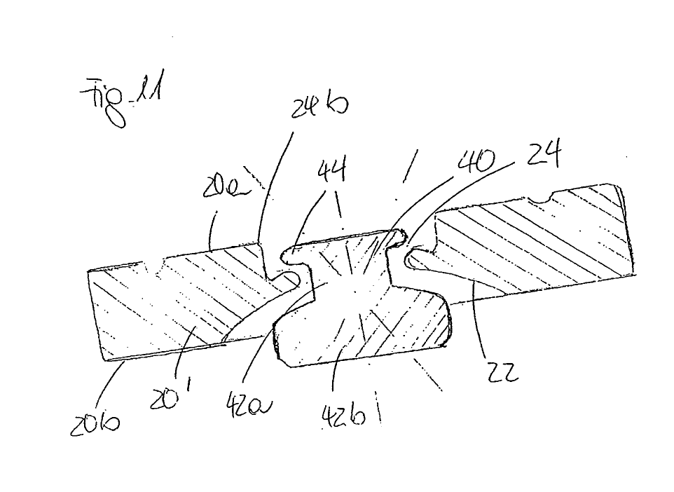

FIG. 7a, another tilted position of the intervertebral disc prosthesis according to FIG. 1a,

FIG. 7b, another tilted position of the intervertebral disc prosthesis according to FIG. 1a,

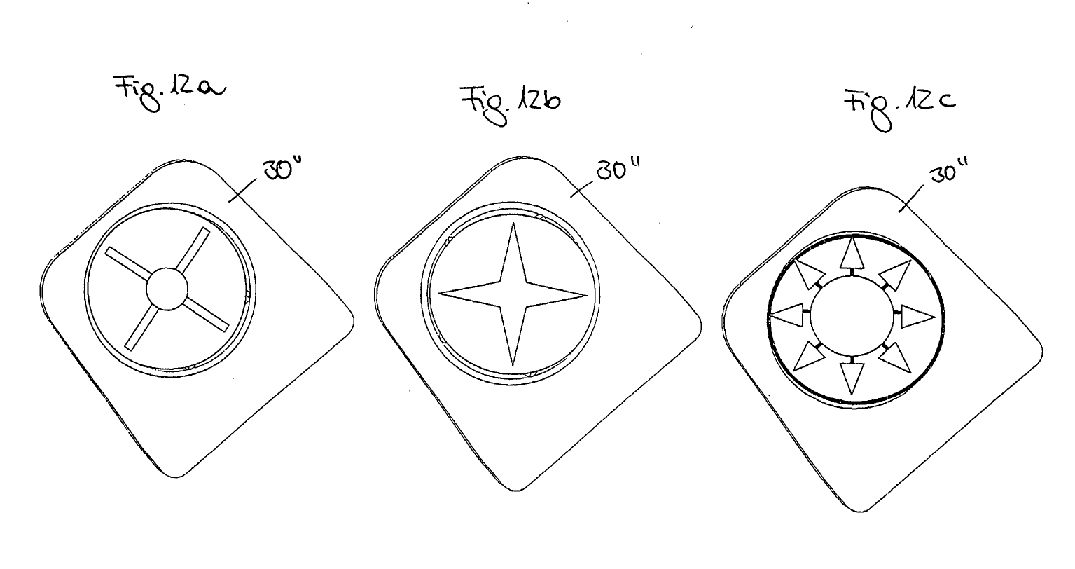

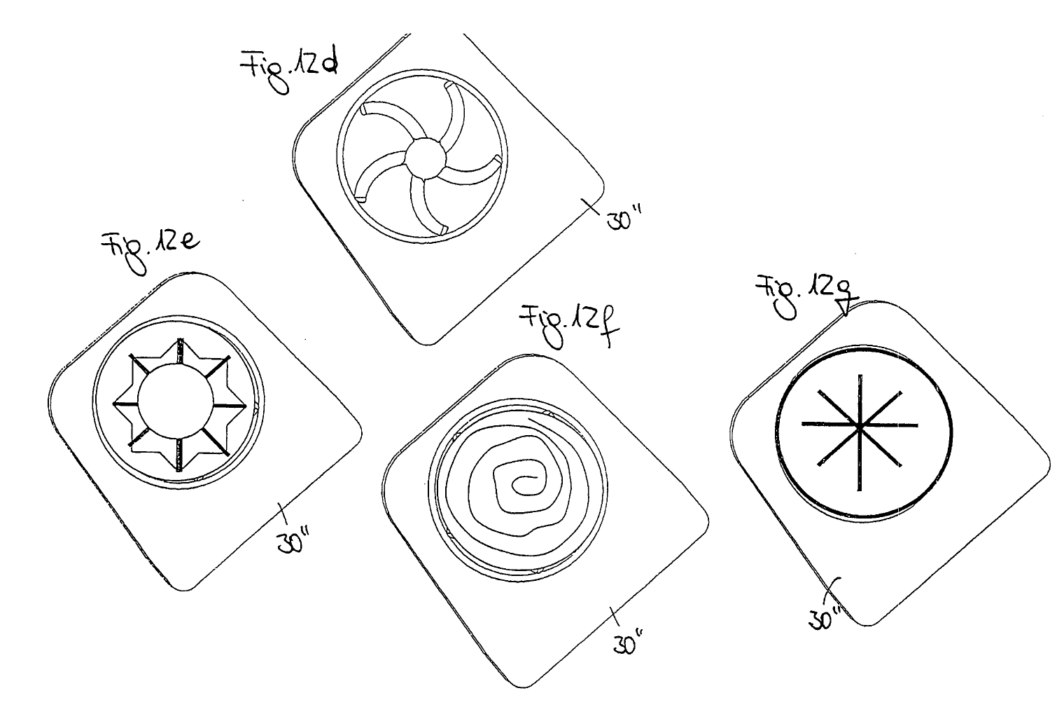

FIG. 8a, a perspective view of the first prosthesis plate of the intervertebral disc prosthesis according to FIG. 1a,

FIG. 8b, the first prosthesis plate according to FIG. 8a in another perspective diagram,

FIG. 8c, an anchoring element and an activation element in perspective view,

FIG. 8d, the anchoring element according to FIG. 8c with mounted activation element,

FIG. 8e, the first prosthesis plate according to FIG. 8a with inserted anchoring element according to FIG. 8c,

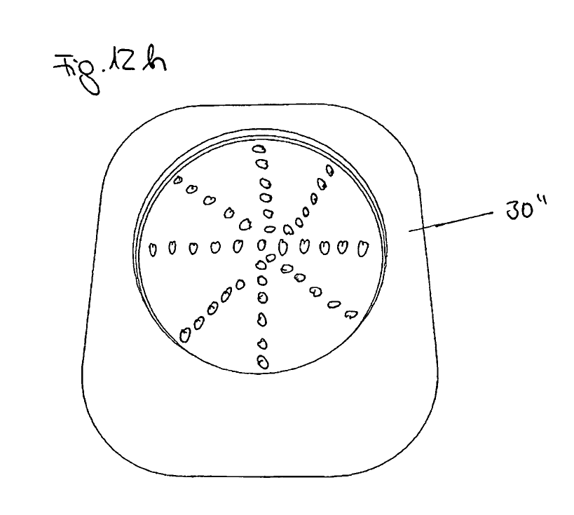

FIG. 9a, a side view of another embodiment example of a second prosthesis plate with an inserted guide element,

FIG. 9b, a perspective view of the second prosthesis plate according to FIG. 9a,

FIG. 9c, another perspective view of the second prosthesis plate according to FIG. 9a,

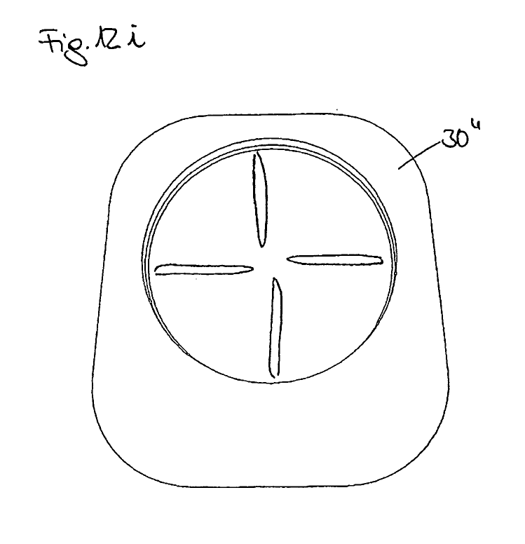

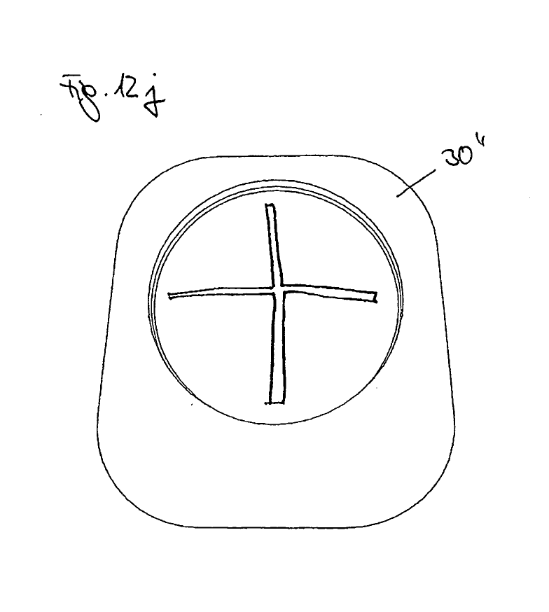

FIG. 10a, a perspective view of a second embodiment example of a first prosthesis plate with inserted guide element and the second prosthesis plate according to FIG. 9a,

FIG. 10b, a top view of the inside of the first prosthesis plate according to FIG. 10a,

FIG. 10c, a perspective view of the first prosthesis plate according to FIG. 10b,

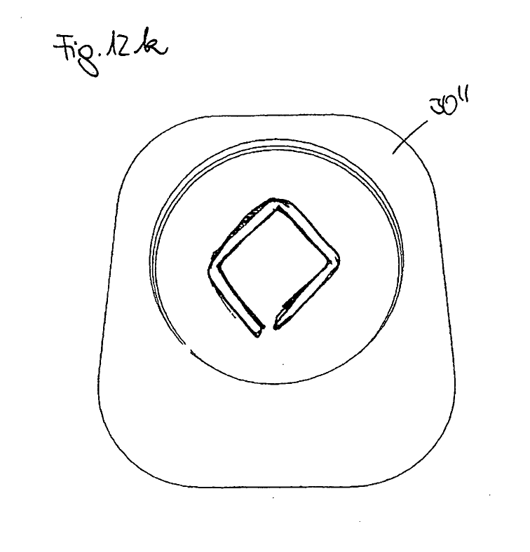

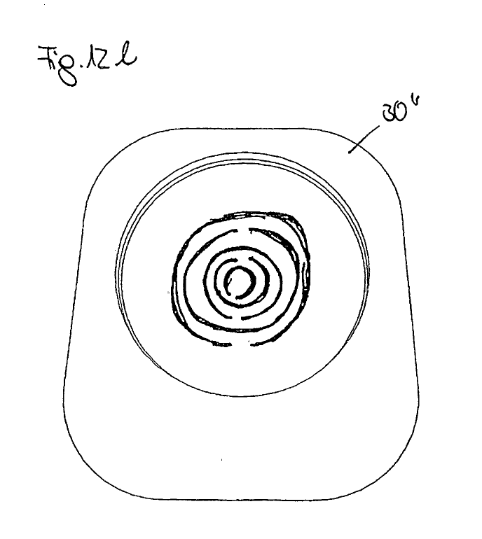

FIG. 11, an axial section through the first prosthesis plate according to FIG. 10c with inserted guide element,

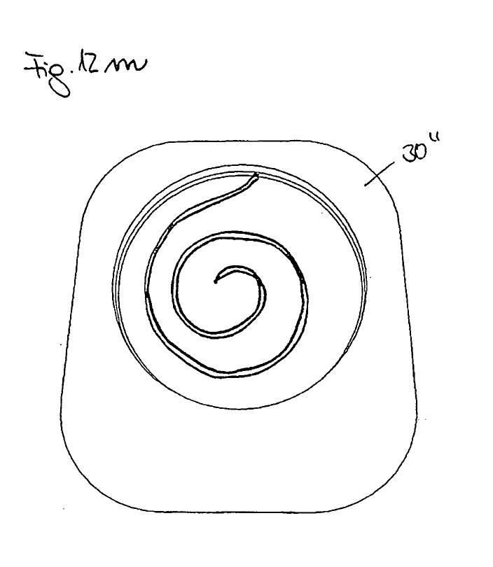

FIG. 12a, a top view of a prosthesis plate with an alternative embodiment of the slots,

FIG. 12b, a top view of a prosthesis plate with another alternative embodiment of the slots,

FIG. 12c, a top view of a prosthesis plate with another alternative embodiment of the slots,

FIG. 12d, a top view of a prosthesis plate with another alternative embodiment of the slots,

FIG. 12e, a top view of a prosthesis plate with another alternative embodiment of the slots,

FIG. 12f, a top view of a prosthesis plate with another alternative embodiment of the slots,

FIG. 12g, a top view of a prosthesis plate with another alternative embodiment of the slots,

FIG. 12h, a top view of a prosthesis plate with another alternative embodiment of the slots,

FIG. 12i, a top view of a prosthesis plate with another alternative embodiment of the slots,

FIG. 12j, a top view of a prosthesis plate with another alternative embodiment of the slots,

FIG. 12k, a top view of a prosthesis plate with another alternative embodiment of the slots,

FIG. 12l, a top view of a prosthesis plate with another alternative embodiment of the slots,

FIG. 12m, a top view of a prosthesis plate with another alternative embodiment of the slots,

FIG. 12n, a top view of a prosthesis plate with another alternative embodiment of the slots,

FIG. 12o, a top view of a prosthesis plate with another alternative embodiment of the slots,

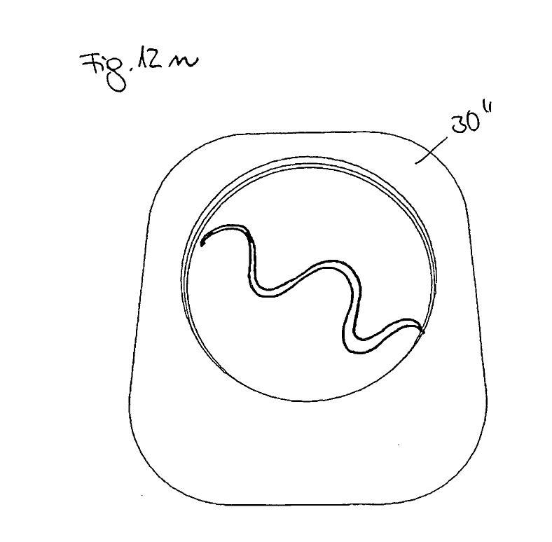

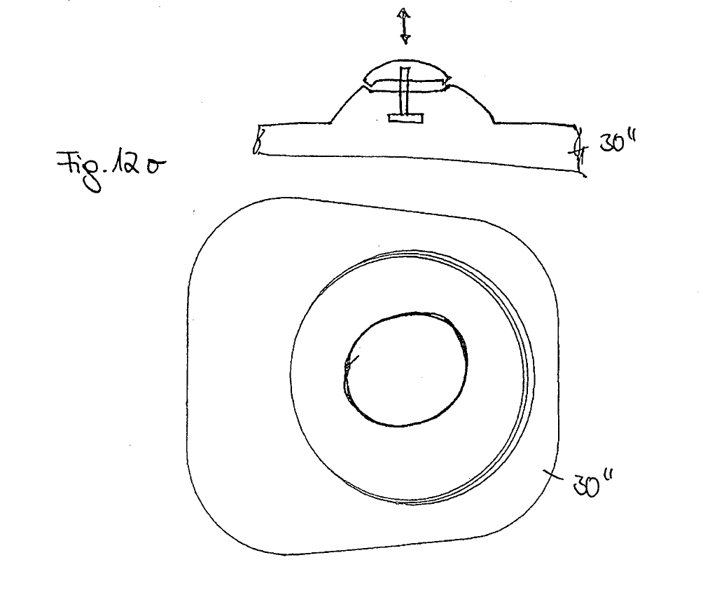

FIG. 13a, an axial section through another embodiment example of an intervertebral disc prosthesis with a first and a second prosthesis plate,

FIG. 13b, an axial section through a slightly modified embodiment example of the intervertebral disc prosthesis according to FIG. 13a,

FIG. 13c, a perspective view of the first prosthesis plate of the intervertebral disc prosthesis according to FIG. 13a, and

FIG. 13d, another perspective view of the first prosthesis plate of the intervertebral disc prosthesis according to FIG. 13a.

FIGS. 1-8 show various views, sometimes only of individual parts, of a first embodiment example of an intervertebral disc prosthesis 10. In all of the figures, the same parts are designated with the same reference symbols wherein, for better visibility, all of the reference symbols are not indicated in all of the figures.

The intervertebral disc prosthesis 10 has a first prosthesis plate 20 and a second prosthesis plate 30. The first prosthesis plate 20 has a side 20a facing the adjacent disc and a side 20b facing the second prosthesis plate 30, while the second prosthesis plate 30 has a side 30a facing the adjacent disc and a side 30b facing the first prosthesis plate 20. If the intervertebral disc prosthesis 10 is inserted into an intermediate space between two adjacent discs, then the sides 20a, 30a contact the adjacent discs, wherein the intervertebral disc prosthesis replaces a defective intervertebral disc that has been previously removed.

Both the first prosthesis plate 20 and also the second prosthesis plate 30 are made from PEEK (polyether ether ketone).

The first prosthesis plate 20 has, on its side 20b facing the second prosthesis plate 30, as to be seen particularly in FIG. 3a, a concave recess 22 that has an approximately ellipsoidal shell segment design, so that the border of the recess 22 lying in the side 20a has an approximately oval or elliptical design. The second prosthesis plate 30 has, on its side 30b facing the first prosthesis plate 20, a convex projection 32 that has an approximately spherical segment design. The projection 32 has a highest point of the curved surface, a pole 32a. The projection 32 also has an approximately circular edge 32b along which the projection 32 borders the side 30b. The projection 32 is hollow and has, starting from the pole 32a, six slots 33 at the same angular spacing, by means of which six approximately triangular and inward curved elastic elements 35 are formed.

The projection 32 is offset towards the back in the sagittal direction from the center of the second prosthesis plate 30 to simulate the natural center of rotation of the intervertebral disc to be replaced. When the intervertebral disc prosthesis 10 is assembled, the convex projection 32 grips into the concave recess 22, by means of which a ball-and-socket joint is formed because the projection 32 is arranged so that it can rotate and tilt in the recess 22 and is also arranged to be movable in a line in the sagittal direction due to the elongated concave recess 22.

The elastic elements 35 of the projection 32 allow the two prosthesis plates 20, 30 to be pressed against each other in the axial direction so that, under high loading of the spine, the movement is damped. The slots 33 are here preferably designed such that the elastic elements 35 bend inward under high loading, such that the elastic elements 35 contact each other with their edges until, in particular, a spherical segment projection 32 of large radius is formed. As a result, it is guaranteed that, even under high loading, the projection 32 is movable with as little friction as possible in the recess 22. In an especially preferred manner, the slots 33 are cut in the projection at an angle to the surface of the projection 32. This can be seen in an especially clear way in FIG. 9c, which will be described below in more detail. By means of the slots 33 cut at an angle, it is not only possible that the edges of the elastic elements 35 contact each other under high loading, but it is also possible for the edges of adjacent elastic elements 35 to contact each other in the radial direction of the projection 32, by means of which the stability of the projection 32 is guaranteed, even under high loading.

FIG. 4-7 show different positions of the two prosthesis plates 20, 30 against one another. FIG. 4a shows the intervertebral disc prosthesis 10 with the first prosthesis plate 20 lifted in the axial direction. FIG. 4b shows the intervertebral disc prosthesis 10 with the first prosthesis plate 20 mounted on the second prosthesis plate 30 in the unloaded state, while FIG. 4c shows the intervertebral disc prosthesis 10 with the first prosthesis plate 20 mounted on the second prosthesis plate 30 under high loading, wherein the two prosthesis plates 20, 30 are pressed together, for example, by approximately 0.5 mm.

FIGS. 5a and 5b show a side view of the intervertebral disc prosthesis 10 from the transverse direction, wherein the first prosthesis plate 20 is shown tilted forward and backward into the maximum positions in the two figures, respectively. With the intervertebral disc prosthesis 10, a flexion and extension motion of approximately ±8° is possible.

In FIGS. 6a and 6b, the intervertebral disc prosthesis 10 is shown in a side view from the transverse direction, wherein the first prosthesis plate 20 is shown shifted forward or backward in a linear direction into the maximum positions relative to the second prosthesis plate 30. This linear shift is enabled by the elongated concave recess 22, wherein a linear motion of up to 1.5 mm is possible.

FIGS. 7a and 7b show the intervertebral disc prosthesis 10 in a side view from the sagittal direction, wherein the first prosthesis plate 20 is shown at the two maximum lateral inclinations relative to the second prosthesis plate 30. With the intervertebral disc prosthesis 10, inclinations up to ±8° are possible. In addition, the intervertebral disc prosthesis 10 allows a rotational radius about the vertical axis of the intervertebral disc prosthesis 10 between ±2°.

To allow the quickest possible and longest lasting biological integration of the intervertebral disc prosthesis 10 in the body of the patient, the prosthesis plates 20, 30 are at least partially provided, particularly on the sides 20a, 20b facing the adjacent discs, with a surface coating, particularly an osteoconductive or osteoinductive coating. For example, a hydroxyapatite coating could be deposited by a plasma spraying method. Alternatively, tricalcium phosphate could be used as a coating material. Such coatings have no metal, particularly no titanium, so that these coatings allow artifact-free MRT imaging.

On the sides 20a, 30a facing the adjacent discs, anchoring elements could be designed on the prosthesis plates 20, 30 in the form of projecting teeth or other projections or surface milling to fix the prosthesis plates 20, 30 on the adjacent discs.

In FIGS. 8a-8e, a preferred anchoring mechanism for anchoring one of the two prosthesis plates, in the present case, the first prosthesis plate 20, is shown in the adjacent disc. The first prosthesis plate 20 has, in its side 20a facing the adjacent disc, two anchoring recesses 26 that run parallel to each other in the sagittal direction and in each of which an anchoring element 50 can be arranged. The anchoring element 50 has an approximately U-shaped design with a first leg 51 and a second leg 55, wherein the second leg 55 is designed longer than the first leg 51. The second leg 55 has a free end 56 on which there is a holding projection 57 angled inward.

With the help of an activation element 60, the anchoring element 50 can be countersunk in the anchoring recess 26 and can be at least partially moved out from this recess. The activation element 60 has an approximately cylindrical base body 61 on which, at one end, there is a forced surface 62 that has an approximately wedge-shaped design. In connection to the forced surface 62, the base body 61 has a peripheral groove 63 with a smaller diameter than the diameter of the base body 61. A transverse slot 64 is diametrically arranged in the base body 61. The transverse slot 64 is extended along one radius through the groove 63 and the forced surface 62 up to the end of the activation element 60 formed by the forced surface 62. As a result, it is possible to mount the activation element 60 on the free end 56 of the second leg 55 of the anchoring element 50 such that the holding projection 57 engages in the transverse slot 64 and the activation element 60 is movable, guided in a linear direction along the free end 56 of the second leg 55 of the anchoring element 50 by means of the projection of the transverse slot 64 into the groove 63 and the forced surface 62.

The first leg 51 of the anchoring element 50 has a free end 52 on which a catch projection 53 extends inward. When the activation element 60 on the second leg 55 is moved to the farthest position in the direction of the free end 56 of the second leg 55, the catch projection 53 lies in front of the forced surface 62. When the activation element 60 is shifted along the second leg 55 in the direction towards the region of the U-shaped anchoring element 50 connecting the two legs 51, 55, the wedge- or cone-shaped forced surface 62 of the activation element 60 has the effect that the catch projection 53 continuously moves away from the second leg 55 with a support edge along it, and the U-shaped anchoring element 50 is bent up.

The anchoring element 50 is arranged in a first sub-area 26a of the anchoring recess 26 of the first prosthesis plate 20 such that the plane of the U of the anchoring element 50 runs perpendicular to the side 20a or 20b. The anchoring element 50 is dimensioned such that it is completely embedded in the anchoring recess 26 in the non-bent state.

The approximately slot-shaped sub-area 26a of the anchoring recess 26 open to the side 20a continues in the sagittal direction towards the back offset in a cylindrical sub-area 26b in which the activation element 60 could be arranged guided so that it could be shifted with a linear motion. When the activation element 60 is pressed along the second leg 55 into the first prosthesis plate 20, the forced surface 62 causes the first leg 51 of the anchoring element 50 to bend up, so that the free end 52 of the first leg 51 projects past the side 20a (cf. FIG. 8e). These projecting regions of the anchoring element 50 are pressed into the adjacent disc body and create, in this manner an anchoring of the prosthesis plate 20 to the adjacent disc. However, when the intervertebral disc prosthesis 10 is inserted into the intervertebral disc space, the anchoring elements 50 are initially countersunk in the anchoring recess 26, so that the insertion of the intervertebral disc prosthesis 10 into the intervertebral disc space is simplified, because the intervertebral disc space must be spread apart less.

To prevent the two prosthesis plates 20, 30 disengaging for non-physical movements or whiplash injuries, instead of the prosthesis plates 20, 30, slightly modified prosthesis plates 20′, 30′ could also be used, between which a guide element 40 is arranged that is described below with reference to FIGS. 9-11.

The guide element 40 has a base body 42 on which one end is a head 44. The head 44 can have a cylindrical, especially with rounded edges, hemispherical, or spherical design. The base body 42 has a pin-like or cylindrical region 42a and an anchoring region 42b, as can be particularly seen in FIG. 11. Here the base body 42 particularly has two parts, wherein the anchoring region 42b is detachably arranged on region 42a.

The base body 42 of the guide element 40 is arranged in a guide recess 34 in the second prosthesis plate 30′, wherein the guide recess 34 essentially extends in the radial direction through the projection 32 and essentially coaxially to the rotational axis of the intervertebral disc prosthesis 10 between the elastic elements 35 and runs in hollow projection 32 up to side 30a of prosthesis plate 30′. As shown in FIG. 9b, side 30a of the guide recess 34 is equipped with an opening 34b so that the guide recess runs in the axial direction through the prosthesis plate 30′. The guide recess 34 here fixes the base body 42 in the horizontal and vertical direction. This essentially occurs in that the anchoring region 42b of the base body 40 is fixed on the inner surface of the side 30a of the prosthesis plate 30′ when the opening 34b of the guide recess is closed by a closure plate, not shown, so that neither a horizontal nor a vertical movement of the guide element 40 is possible relative to the second prosthesis plate 30′.

FIGS. 10a, 10b, and 10c show the first prosthesis plate 20′ that differs from the previously described first prosthesis plate 20 in that an additional guide recess 24 is arranged in the recess 22. In the articulation surface of the recess 22, the guide recess 24 forms an approximately oval or elliptical surface and extends into the first prosthesis plate 20′ as an undercut recess. As is to be taken from FIGS. 10a and 11 particularly, the guide recess 24 extends in the axial direction through the prosthesis plate 20′ and has an opening 24b in the side 20a.

The head 44 of the guide element 40 is formed in the guide recess 24 such that it can be moved in the horizontal and vertical directions in the undercut guide recess 24. For the rotation, tilting, or linear movement of the projection 32 in the recess 22, the head 44 moves in the undercut guide recess 24 of the recess 22 of the first prosthesis plate 20′ and thus barely prevents movement. Under high loading, the guide element 40 could move in the axial direction in the guide recess 24 wherein, however, a stop for a maximum flattening of the intervertebral disc prosthesis 10 is formed by the length of the base body 42 of the guide element 40. The guide element 40, however, prevents complete detachment of the first prosthesis plate 20′ from the second prosthesis plate 30′ for relative movements that are too large.

Thus, for inserting the guide element 40 into the second prosthesis plate 30′, the guide element 40 is disassembled into two parts. From the side 20a of the first prosthesis plate 20′, the head 44 with the region 42a arranged on this head is inserted in advance through the opening 24b into the guide recess 24 until the free end of the region 42a is arranged in the concave recess 22. Then, the anchoring region 42b is arranged on region 42a. Then closure plates are mounted both on the opening 24b of the guide recess 24 of the first prosthesis plate 20′ and also on the opening 34b of the guide recess 34 of the second prosthesis plate 30′ to close the openings 24b, 34b.

The slots 33 of the second embodiment example of the prosthesis plate 30′ are curved starting from the pole 32a of the projection 32 in the peripheral direction running toward the edge 32b.

In FIGS. 12a-12o, a prosthesis plate 30″ with a convex projection is shown with different embodiments of slots promoting the spring effect of the convex projection. This form of the slots could alternatively also be arranged in a concave recess.

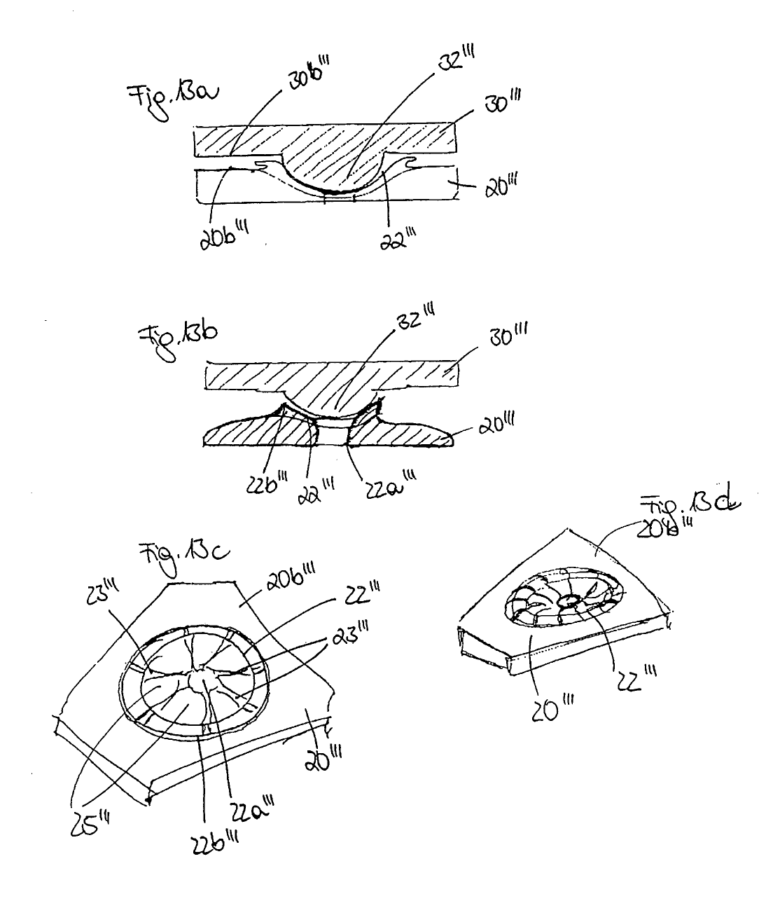

For example, the different embodiments have slots that start from the pole of the convex projection and run in the direction of the edge (cf. FIGS. 12a, 12b, 12d, 12g, 12j), partially tapering (cf. FIG. 12b) or having a curved profile (cf. FIG. 12d). FIG. 12c shows a convex projection with a larger opening around the pole and bordering approximately triangular-shaped slots. In the embodiment example, according to FIG. 12e, there is an approximately star-shaped recess as a slot. FIGS. 12f and 12m show a spiral-shaped, curved slot, while in FIG. 121 several concentric slots are shown wherein the slots, however, do not form a closed ring. In FIG. 12h, several series of holes are arranged in the convex projection. FIG. 12i shows four slots that run toward the edge of the convex projection and that, however, are not connected to each other like the slots in FIG. 12j for example. FIG. 12k shows a slot that runs around the pole, has an approximately square shape, and that, however, also does not form a closed ring. FIG. 12n shows a slot that has several curves, a particularly wave-shaped profile, and that extends from one edge of the convex projection past the pole approximately up to the opposite edge of the projection.

In the embodiment examples of FIGS. 12a-12n, the slots are each formed so that the convex projection has a one-piece design. FIG. 12o shows an example for a two-part convex projection in which the pole is separated by a slot running around the pole and above which an element that makes possible axial movement of the pole cap relative to the prosthesis plate is arranged between the pole cap and the remaining part of the convex projection, particularly prosthesis plate 30″.

In FIGS. 13a-13d, an embodiment example of an intervertebral disc prosthesis is shown with a first prosthesis plate 20′″ that has on side 20b′″ a concave recess 22′″ and a second prosthesis plate 30′″ that has on a side 30b′″ facing the first prosthesis plate 20′″ a convex, approximately spherical segment projection 32′″ that engages in the concave recess 22′″, in which the concave recess 22′″ has at least one elastic element 25′″. The concave recess 22′″ has an essentially spherical shell design and has a pole 22a′″ and an edge 22b′″.

FIGS. 13a and 13b each show an axial section through the intervertebral disc prosthesis wherein slight differences consist in that, in the embodiment example, according to FIG. 13a, edge 22b′″ of the recess 22′″ is bent outward and forms an undercut peripheral edge 22b′″, while edge 22b′″ of the recess 22′″ of the embodiment example in FIG. 13b is not bent outward.

FIGS. 13c and 13d show two perspective views of the first prosthesis plate 20′″ of the intervertebral disc prosthesis according to FIG. 13a, in which it can be seen that, in recess 22′″, the elastic elements 25′″ are formed such that several slots run to the edge 22b′″ starting from the pole 22a′″ of the recess 22′″. The slots 23′″ could also assume other designs, as shown in FIGS. 12a-12o for example. The convex projection 32′″ of the second prosthesis plate 30′″ could have a solid or hollow construction and be pressed deeper into the recess 22′″ against the spring force of the elastic elements 25′″ under high axial loading of the intervertebral disc prosthesis.

### LIST OF REFERENCE SYMBOLS

10 Intervertebral disc prosthesis
20 First prosthesis plate
20′ First prosthesis plate
20′″ First prosthesis plate
20a Side
20b Side
20b′″ Side
22 Recess
22b′″ Recess
22a′″ Pole
22b′″ Edge
23′″ Slot
24 Guide recess
24b Opening
25′″ Elastic element
26 Anchoring recess
26a Sub-area
26b Sub-area
30 Second prosthesis plate
30′ Second prosthesis plate
30″ Second prosthesis plate
30′″ Second prosthesis plate
30a Side
30b Side
30b′″ Side
32 Projection
32′″ Projection
32a Pole
32b Edge
33 Slot
34 Guide recess
34b Opening
35 Elastic element
40 Guide element
42 Base body
42a Region
42b Anchoring region
44 Head
50 Anchoring element
51 First leg
52 Free end
53 Catch projection
55 Second leg
56 Free end
57 Holding projection
60 Activation element
61 Base body
62 Forced surface
63 Groove
64 Transverse slot

1. Intervertebral disc prosthesis (10) with a first prosthesis plate (20) and a second prosthesis plate (30) wherein the first prosthesis plate (20) has on its side (20b) facing the second prosthesis plate (30) a concave recess (22) in which engages a convex projection (32) arranged on the side (30b) of the second prosthesis plate (30) facing the first prosthesis plate (10) characterized in that the convex projection (32) and/or the concave recess has at least one elastic element (35).

2. Intervertebral disc prosthesis according to claim 1, characterized in that the convex projection (32) and/or the concave recess has a hollow construction.

3. Intervertebral disc prosthesis according to claim 1, characterized in that the convex projection (32) and/or the concave recess has at least one slot (33).

4. Intervertebral disc according to claim 1, characterized in that the one or more slots (33) are cut at an angle to the surface of the convex projection (32) or the concave recess.

5. Intervertebral disc prosthesis according to claim 1, characterized in that the convex projection (32) and/or the concave recess has several slots (33) that start from its pole (32a) and that run in the direction of the edge (32b) of the convex projection (32).

6. Intervertebral disc prosthesis according to claim 1, characterized in that the recess has a spherical or ellipsoidal shell segment design.

7. Intervertebral disc prosthesis according to claim 1, characterized in that the projection (32) has a spherical segment design.

8. Intervertebral disc prosthesis according to claim 1, characterized in that the projection (32) is arranged offset towards the back in the sagittal direction from the center of the prosthesis plate (30).

9. Intervertebral disc prosthesis according to claim 1, characterized in that a guide element (40) that has a base body (42) and a head (44) arranged on one end of the base body (42) is arranged between the first prosthesis plate (20) and the second prosthesis plate (30), wherein the base body (42) engages in a guide recess (34) in the second prosthesis plate (30) and the head (44) engages in a guide recess (24) in the first prosthesis plate (20).

10. Intervertebral disc prosthesis according to claim 9, characterized in that the guide recess (34) of the second prosthesis plate (30) is arranged in the projection (32), particularly between the elastic elements (35).

11. Intervertebral disc prosthesis according to claim 9, characterized in that the guide recess (34) of the second prosthesis plate (30) fixes the guide element (40) in the horizontal and vertical direction relative to the second prosthesis plate (30).

12. Intervertebral disc prosthesis according to claim 9, characterized in that the guide recess (24) of the first prosthesis plate (20) is arranged in recess (22).

13. Intervertebral disc prosthesis according to claim 9, characterized in that the guide recess (24) of the first prosthesis plate (20) allows movement of the guide element (40) in the horizontal and vertical direction.

14. Intervertebral disc prosthesis according to claim 9, characterized in that the guide recess (24) of the first prosthesis plate (20) has an oval or elliptical design, wherein the longitudinal axis of the guide recess (24) runs in the sagittal direction.

15. Intervertebral disc prosthesis according to claim 1, characterized in that the first prosthesis plate (20) and/or the second prosthesis plate (30) is made from PEEK.

16. Intervertebral disc prosthesis according to claim 1, characterized in that the side (20a, 30a) facing the disc on the first prosthesis plate (20) and/or the second prosthesis plate (30) has an osteoconductive and/or an osteoinductive coating.
